(19) United States
(12) Patent Application Publication
(10) Pub. No.: US 2008/0287937 A1
(43) Pub. Date: Nov. 20, 2008

(54) Surgical Instrument for Illuminating and Monitoring a Surgical Site

(75) Inventor: Anthony J. Melkent, Memphis, TN (US)

Correspondence Address: COATS & BENNETT/MEDTRONIC, 1400 CRESCENT GREEN, SUITE 300, CARY, NC, 27518, US

(73) Assignee: Warsaw Orthopedic, Inc., Warsaw, IN (US)

(21) Appl. No.: 11/748,789

(22) Filed: May 15, 2007

(51) Int. Cl. A61B 18/00

(52) U.S. Cl. 606/14

(57) ABSTRACT

The present application discloses embodiments of a surgical instrument to monitor a surgical site within a patient. In one embodiment, the instrument includes an elongated extender including a distal end and a proximal end. A body may be attached to the distal end of the extender. A plurality of light elements may be permanently attached to the body. The light elements may be spaced around the body and form a perimeter to provide dispersed light to the surgical site. An optical input member may be permanently attached to the body. The optical input member may include a distal end that faces outward away from the extender and towards the surgical site.

### BACKGROUND

The present application is directed to methods and devices for viewing a surgical site and, more particularly, to devices that provide a dispersed lighting arrangement and optical input source for visualization of the surgical site.

Endoscopes provide for minimally-invasive monitoring of a surgical site. The endoscopes include an elongated, thin tube sized to be inserted into a patient and moved to the surgical site. Fiber-optical instruments or a series of rigid lenses and a viewing mechanism are included within the tube for observation either with the naked eye or an attached camera. One or more light elements are further positioned within the tube for distributing light to the surgical site.

The light elements provide for adequate lighting of the surgical site to allow the surgeon to perform the surgical procedure. However, there are limits to the amount of light and/or the direction of the light provided by the light elements. The light elements are often positioned such that light is dispersed distal to the end of the tube. Further, the light is typically limited to the direction that the light element is aimed. This arrangement optimizes the light for the fiber-optical instruments or lenses and viewing mechanism so the surgeon is provided with an adequate image to perform the surgical procedure.

A drawback of these endoscopes is the highly-directed light elements are not optimal for viewing with the naked eye or with loupes, which would occur via the primary surgeon and is preferred due to the limitations of current endoscopes including image quality (e.g., 2D vs. 3D image, motion magnification of hand/tool movements, etc). The optical image observed by the endoscopes and displayed on a monitor is an advantage, though, as it allows observation by other persons in the operating room such as staff or surgeons in training.

Some prior procedures have included lighting the surgical site through a second portal into the patient. However, the second portal increases the invasiveness of the procedure and may result in various issues including additional discomfort to the patient, increased risk of infection, and a longer recovery time

### SUMMARY

The present application discloses embodiments of a surgical instrument to monitor a surgical site within a patient. The instrument may allow for optimal lighting for the primary surgeon via direct vision, while compromising on the image that is presented on the monitor for the other persons in the operating room. In one embodiment, the instrument includes an elongated extender including a distal end and a proximal end. A body may be attached to the distal end of the extender. A plurality of light elements may be permanently attached to the body. The light elements may be spaced around the body and form an perimeter to provide dispersed light to the surgical site. An optical input member may be permanently attached to the body. The optical input member may include a distal end that faces outward away from the extender and towards the surgical site.

### BRIEF DESCRIPTION OF THE DRAWINGS

FIG. 1 is a schematic diagram of a surgical viewing system according to one embodiment.

FIG. 2 is a perspective view of a surgical instrument operatively connected to a light source and a viewer according to one embodiment.

FIG. 3 is an end view of a surgical instrument according to one embodiment.

FIG. 4 is a partial perspective view of an optical input member according to one embodiment.

FIG. 5 is a side schematic view of an optical input member according to one embodiment.

FIG. 6 is an end view of a surgical instrument according to one embodiment.

FIG. 7 is a perspective view of a portion of a surgical instrument positioned within a retractor according to one embodiment.

FIG. 8 is an end view of a surgical instrument according to one embodiment.

### DETAILED DESCRIPTION

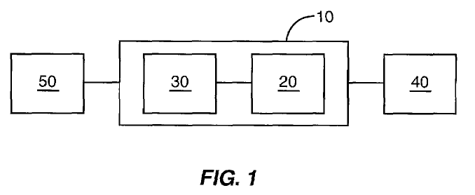

The present application is directed to embodiments of a surgical instrument for illuminating and monitoring a surgical site. FIG. 1 illustrates a schematic illustration of one embodiment of the device 10 included within a surgical viewing system. The device 10 includes a plurality of lighting elements 20 configured to provide dispersed lighting to the surgical site. An optical input member 30 is associated with and utilizes the dispersed lighting. The lighting elements 20 and optical input member 30 are sized to be inserted into the patient and delivered to the surgical site. The device 10 is operatively connected to a light source 40 that provides light through the lighting elements 20 to the surgical site. The device 10 is operatively connected to a viewer 50 for monitoring of the surgical site by various observers of the surgical procedure.

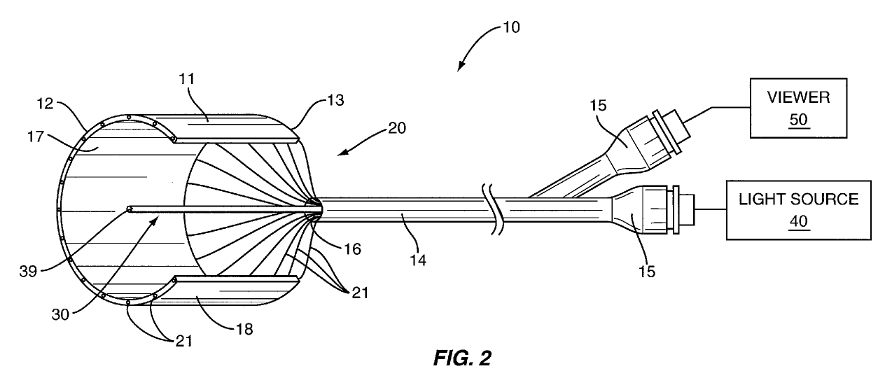

FIG. 2 illustrates one embodiment of the surgical device 10. The device 10 generally includes a body 11 and an elongated extender 14. In one embodiment, the width of the body 11 is greater than the extender 14, and the extender 14 includes a greater length. The body 11 is curved and forms a substantially C-shape with a hollow interior that forms a working channel. A gap is formed between the sides of the body 11 and extends along the length. Body 11 includes a distal end 12 that faces towards the surgical site with a proximal end 13 facing towards the extender 14.

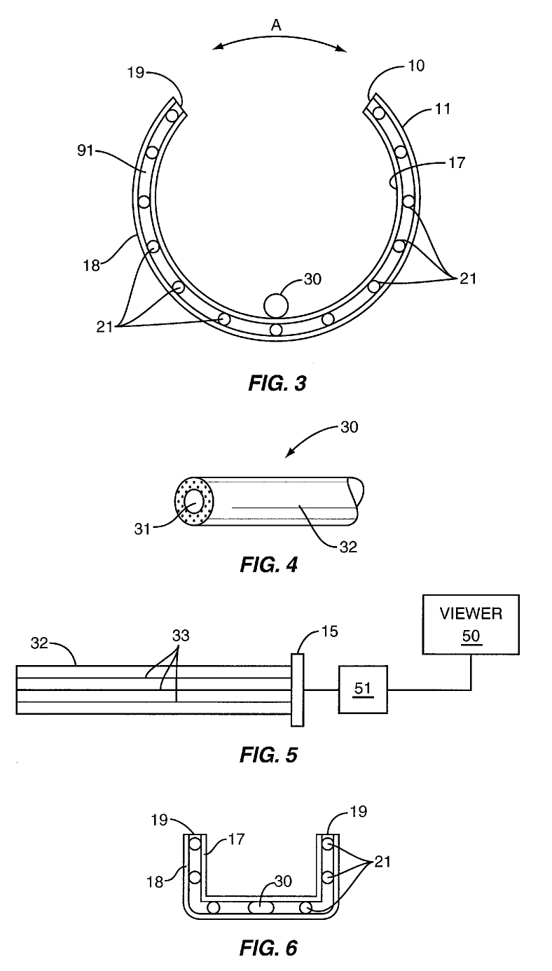

In one embodiment as illustrated in FIG. 3, the body 11 includes a two-ply construction formed by an inner member 17 and an outer member 18. The members 17, 18 may be coupled to one another at their lateral edges 19. The gap between the lateral edges 19 may vary depending upon the context of use. The members 17, 18 are spaced apart forming a passage 91 to position a distal end of light elements 21 and/or optical input member 30 as will be explained in more detail below. In one embodiment, an epoxy is applied within the passage 91 between the members 17, 18. Epoxy functions to maintain the members 17, 18 connected together. Epoxy may also protect and position the light elements 21 and/or optical input member 30.

The extender 14 is operatively connected to the body 11 and provides a structure for insertion into the patient. In one embodiment, the body 11 is attached to a distal end of the extender 14. In another embodiment, the body 11 is spaced away from the distal end. Extender 14 includes a hollow interior space 16 that forms a conduit for containing the light elements 21 and optical input member 30. One or more couplings 15 are positioned at a proximal end for attachment with the light source 40 and viewer 50. In one embodiment as illustrated in FIG. 2, a first coupling 15 is for the light source 40, and a second coupling 15 for the viewer 50. The extender 14 includes an elongated shape such that the body 11 may be positioned at the surgical site with the couplings 15 remaining at a point exterior to the patient. Extender 14 may be substantially rigid, or may be flexible.

The light elements 20 are configured to receive light from the light source 40 and direct the light to the surgical site. In one embodiment, the light elements 21 include a first end that is operatively connected to the coupling 15 to receive light from the light source 40. The light elements 20 extend through the length of the extender 14 and into the body 11 where they terminate at the distal end 12. In another embodiment, the first ends of the light elements 21 are spaced from the coupling 15. Light is transferred through the coupling 15 and into the extender 14 prior to being received within the light elements 21.

In one embodiment as illustrated in FIGS. 2 and 3, the light elements 21 extend within the passage 91 formed between the inner wall 17 and outer wall 18 of the body 11 and terminate at the distal end 12. The light elements 21 emit light from their distal ends to illuminate the surgical site. As illustrated in FIG. 3, the light elements 21 are spaced around the body 11 forming a perimeter to create a dispersed lighting arrangement to the surgical site. The perimeter formed by the light elements 21 directs light from a variety of different directions to the surgical site. The perimeter may be formed around an entirety of the body 11, or a limited section.

The optical input member 30 utilizes the dispersed lighting provided through the light elements 21 for visual monitoring of the surgical site. The optical input member 30 includes an elongated shape that extends from the coupling 15, through the extender 14, and along the body 11. Optical input member 30 is configured with the coupling 15 to feed the image to the viewer 50. The optical input member 30 in this embodiment does not include an independent light source, but rather relies on the light provided through the lighting elements 20. This light from the lighting elements 20 may not be adequate for precise visualization such as that required for endoscopic surgery, but is adequate for monitoring by other, non-surgical persons.

FIG. 4 illustrates one embodiment of a distal end of the optical input member 30. The optical input member 30 includes an objective lens 31 positioned within a sheath 32. In one embodiment, the objective lens 31 includes an array of one or more fibers consisting of strands of glass or plastic that transmits the light through repeated internal reflection along the length. This light is directed to the viewer 50 for image formation and visualization. The embodiment of FIG. 4 includes a single objective lens 31.

FIG. 5 is a schematic illustration of another embodiment of an optical input member 30. This embodiment features an electronic configuration with one or more optical fibers 33 extending along the length of the optical input member 30. A processor 51 is positioned between the coupling 15 and viewer 50. The processor 51 includes a charge-coupled device that converts the optical image to electric signals, and a video processor that converts analog electric signals to digital and processes them to video signals that are displayed on the viewer 50. In one embodiment as illustrated in FIG. 5, three optical fibers 33 are positioned each for carrying one of red, blue, and green light.

In one embodiment as illustrated in FIG. 2, the optical input member 30 is positioned along an inner surface of the inner wall 17. This positioning uses a minimum of space as will be explained in detail below. This positioning may also afford protection to the optical input member 30 as the body 11 acts as a shield to prevent damage to the optical input member 30. FIG. 6 illustrates another embodiment with the optical input member 30 positioned between the inner wall 17 and outer wall 18 of the body 11. This positioning may also minimize the space taken by the optical input member 30 and also provide protection.

In one embodiment, the distal end of the optical input member 30 may be substantially aligned with the distal end 12 of the body 11. This positioning prevents the formation of shadows caused with the light elements 21 or block the view of the optical input member 30. In another embodiment, the distal end is recessed inward away from the distal end 12 of the body 11. The recessed position protects the optical input member 30 and may also prevent the distal end from being positioned too close to the surgical site which may prevent adequate viewing by the observers. The optical input member 30 may also extend outward beyond the distal end 12 of the body 11.

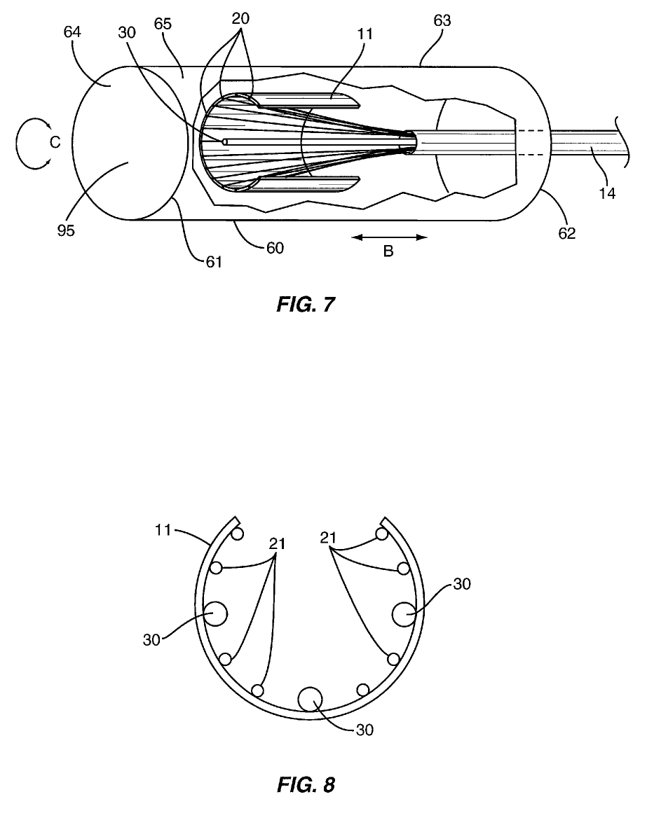

In one embodiment as illustrated in FIG. 7, the device 10 is used in combination with a retractor 60. Retractor 60 includes a body 63 that extends between a distal end 61 and a proximal end 62. The body 63 defines a working channel 95 that extends between the ends 61, 62. The working channel 95 is sized to receive one or more surgical instruments during the surgical procedure.

In one embodiment, the retractor 60 is inserted into the patient over the last of one or more tissue dilators and/or guidewires sequentially positioned one around the other to gradually retract the tissue and skin of the patient. With the retractor 60 positioned through the skin and tissue, the dilators are removed to provide access to the surgical site through the working channel 95.

The body 63 includes an inner wall surface 64 and an outer wall surface 65. The device 10 is positioned with the body 11 in the working channel 95 such that outer wall 18 is positioned adjacent the inner wall surface 64. The distal ends of the light elements 20 are dispersed at least partially around the inner wall surface 64 to provide greater illumination of the surgical site. In one embodiment, the light elements 20 extend around about 50 percent or more of the inner perimeter of retractor body 63. In one embodiment, the light elements 20 are spaced in a perimeter that extends about 90 degrees along the body 63. The size of the perimeter provides for diffuse lighting of the surgical site that may be received by the optical input member 30. In other embodiments, the perimeter may range from about 10° to about 360°.

When the body 11 is inserted, the distal end of the optical input member 30 is facing outward through the open end of the retractor body 63. This provides for the member 30 to obtain detailed images of the surgical site.

Body 11 may be deformable or manipulated to various configurations by moving the lateral edges 19 as indicated by arrow A in FIG. 3. In this manner, the positioning of the light elements 20 and optical input member 30 may be altered, if needed, to conform with the inner wall surface 64 of the retractor body 63. For example, the body 11 can be reduced in size and positioned in working channel 95 by moving lateral edges 19 toward one another. Once inserted in working channel 95, edges 19 may be returned toward their original configuration, if necessary, causing outer wall 18 to contact inner wall surface 64 in frictional engagement therewith.

The body 11 may be moved axially, as indicated by arrow 80 in FIG. 7, to adjust the positioning of lighting elements 20 and optical input member 30 in the working channel 95 while maintaining frictional engagement with the inner wall surface 64 of the retractor body 63. Body 11 may also be rotated about inner wall surface 64, as indicated by arrow C in FIG. 7, to adjust the positioning of light elements 20 and optical input member 30 about the working channel 95 while maintaining frictional engagement with inner wall surface 64. By providing a readily adjustable body 11 that maintains engagement with the retractor body 63 during adjustment, the surgeon can reposition the location and direction of the emitted light and the optical feed as needed without removing body 11 from the working channel 95, or without the risk of the body 11 falling into the surgical site. In another embodiment, the body 11 is fixedly connected to the retractor body 63.

The body 11 may include a shape that corresponds to the shape of working channel 95, and thus the working channel 95 remains substantially unobstructed during the surgical procedure. Surgical instruments, implants and the like can be positioned through the working channel 95 while the body 11 remains engaged with retractor body 63. Further, the radially dispersed light elements 20 provide a multitude of directions and locations from which light can be emitted, reducing the chance of completely or substantially obstructing the emitting light during the surgical procedure.

FIG. 8 illustrates another embodiment with multiple optical input members 30. The optical input members 30 are spaced apart along the body 11. The multiple optical input members 30 may provide for viewing of the surgical site from different angles. Further, the optical input members 30 provide some redundancy in the system in the event any of the optical input members 30 malfunctions or is somehow obstructed. The number and positioning of the optical input members 30 may vary depending upon the context of use.

Body 11 may include various different shapes. In one embodiment as illustrated in FIG. 2, body 11 is substantially C-shaped. In another embodiment, body 11 is a continuous member (i.e., without a gap). Body 11 may include various shapes including but not limited to circular, oval, rectangular, and polygonal. Body 11 may further be constructed to be deformable such that the shape changes such as during insertion into the retainer 60.

In one embodiment, the optical input member 30 and the light elements 20 are permanently connected to the body 11 such that they are not adjustable axially along the length. This positioning provides for the optical input member 30 to be positioned at a known distance from the light elements 20 to obtain adequate diffuse lighting for the signal returned through the optical input member 30. In one embodiment, the distal ends of the light elements 20 and optical input member 30 are aligned along a common plane.

Embodiments of a surgical device with a plurality of lights are disclosed in U.S. Patent Application Publication 2004/0143169 herein incorporated by reference in its entirety. Another embodiment is the METRx RADIANCE Illumination System available from Medtronic Sofamor Danek of Memphis Tenn., which is also herein incorporated by reference in its entirety.

In one embodiment, the surgical site is adjacent to a spinal column segment. The surgical site may include, for example, paraspinous tissue, the bony tissue of one or more vertebral members, and annulus tissue of a disc space between the vertebral members. The present devices and methods may be used for minimally invasive surgery such as a laminotomy, laminectomy, foramenotomy, facetectomy, discectomy, positioning of interbody implants, positioning of intrabody implants, bone cutting and removal, tissue cutting and removal, and nerve root and tissue retraction, for example.

Light source 40 may be a variety of devices capable of generating and/or transmitting light to the extender 14. The extender 14 may include one or a combination of fiber optic cables, including plastic fiber optic cables, wires or other transmission device or devices capable of transmitting light between light source 40 and the device 10.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present methods and devices may be carried out in other specific ways than those herein set forth without departing from the scope and essential characteristics. The present embodiments are, therefore, to be considered in all respects as illustrative and not restrictive, and all changes coming within the meaning and equivalency range of the appended claims are intended to be embraced therein.

What is claimed is:

1. A surgical instrument to monitor a surgical site within a patient comprising:
an elongated extender including a distal end and a proximal end;
a body with a hollow interior section attached to the distal end of the extender;
a plurality of three or more light elements permanently attached to the body and being spaced around the body to form a perimeter of at least about 10 degrees to provide diffused light to the surgical site; and
an optical input member permanently attached to the body and including a distal end that faces outward away from the extender and towards the surgical site.

2. The instrument of claim 1, wherein the body includes a cylindrical shape with a gap extending along a length.

3. The instrument of claim 1, wherein the extender is hollow and includes an interior space to contain sections of the plurality of light elements and the optical input member.

4. The instrument of claim 3, further including a coupler positioned at the proximal end of the extender, the coupler constructed to operatively connect to a viewer and a light source.

5. The instrument of claim 1, wherein the body includes a two-ply construction with an outer wall and an inner wall that are spaced apart to form a passage therebetween.

6. The instrument of claim 5, wherein the plurality of light elements are positioned within the passage formed between the inner and outer walls.

7. The instrument of claim 5, wherein the optical input member is positioned within the passage formed between the inner and outer walls.

8. The instrument of claim 1, wherein the optical input member includes a sheath that extends around a lens.

9. A surgical instrument to monitor a surgical site within a patient comprising:
an elongated extender;
a body attached to one end of the extender;
a plurality of light elements permanently attached to the body and each including a distal end that faces outward away from the extender, the plurality of light elements being spaced around a majority of the body and forming a perimeter; and
an optical input member permanently attached to the body and including a distal end that faces away from the extender;
the distal ends of the optical input member and the plurality of light elements each being axially constrained within a common axial plane along the body.

10. The instrument of claim 9, wherein the optical input member is positioned within an interior of the perimeter formed by the plurality of light elements.

11. The instrument of claim 9, wherein the optical input member is aligned with the perimeter formed by the plurality of light elements

12. The instrument of claim 9, wherein the perimeter extends at least about 10 degrees.

13. The instrument of claim 9, wherein the body includes a C-shaped cross-sectional shape with a gap formed between a pair of opposing arms and extending along a length.

14. The instrument of claim 13, wherein the body is constructed of a flexible material to adjust a size of the gap.

15. A surgical instrument to monitor a surgical site within a patient comprising:
an elongated extender including a distal end and a proximal end;
a substantially hollow body attached to the distal end of the extender, the hollow body including a working channel within an interior space;
a plurality of outputs to provide light to the surgical site, the plurality of outputs being permanently attached to the body and being spaced around the body to form a perimeter of at least about 10 degrees; and
an optical input to receive images from the surgical site that are illuminated by the plurality of outputs, the optical input being fixed to the body.

16. The instrument of claim 15, wherein distal ends of the plurality of outputs and optical input are substantially aligned within a common axial plane along the body.

17. The instrument of claim 15, wherein each of the plurality of inputs is substantially identical.

18. The instrument of claim 15, wherein the body includes a greater width than the extender.

19. The instrument of claim 15, wherein the extender includes a greater length than the body.

20. A surgical instrument to monitor a surgical site within a patient comprising:
an elongated extender including a distal end and a proximal end;
a body attached to the distal end of the extender, the body including a passage formed between an inner wall and an outer wall;
a plurality of light elements permanently attached to the body and being positioned within the passage, the plurality of light elements being spaced around the body to form a perimeter to provide light to the surgical site; and
an optical input member permanently attached to the body and including a distal end that faces outward away from the extender and towards the surgical site.

21. The instrument of claim 20, wherein the perimeter formed by the plurality of light elements extends at least about 10 degrees.

22. The instrument of claim 20, wherein distal ends of the plurality of light elements and the optical input member are substantially aligned within a common plane.

23. The instrument of claim 20, wherein the optical input member is positioned within the passage formed between the inner and outer walls of the body.

24. The instrument of claim 20, wherein the optical input member is attached to the inner wall of the body.
